(19) United States
(12) Patent Application Publication
(10) Pub. No.: US 2006/0141584 A1
(43) Pub. Date: Jun. 29, 2006

(54) Pretreatment of biomass for ethanol production

(76) Inventors: David Litzen, Rapid City, SD (US); David Dixon, Rapid City, SD (US); Patrick Gilcrease, Rapid City, SD (US); Robb Winter, Rapid City, SD (US)

Correspondence Address: Gene R. Woodle, 3516 Woodle Drive, Rapid City, SD, 57702, US

(21) Appl. No.: 11/320,115

(22) Filed: Dec. 27, 2005

Related U.S. Application Data
(60) Provisional application No. 60/640,146, filed on Dec. 28, 2004.

(51) Int. Cl. C12P 19/02

(52) U.S. Cl. 435/105

(57) ABSTRACT

A pretreatment of biomass for ethanol production is disclosed. The pretreatment of biomass such as sawdust or wood chips by processing such biomass through an extruder results in a greatly increased recovery of sugar using hydrolysis.

### RELATED APPLICATIONS

This application relies for priority upon the Provisional Patent Application filed by David Litzen et al. entitled Pretreatment of Biomass for Ethanol Production, Serial Number 60/640,146, filed Dec. 28, 2004.

### BACKGROUND OF THE INVENTION

1. Field of the Invention

The present invention relates generally to the production of ethanol from biomass such as wood chips or other lignocellulosic feed stocks and more specifically to the pretreatment of biomass to improve ethanol production efficiency.

2. Background Information

Largely because of the cost and fluctuations in supply, there is a worldwide interest in finding replacements or substitutes for naturally occurring oil. Much of the interest in replacements for oil is focused upon the production and use of ethanol. The use of ethanol as a part of the supply of motor fuel, for instance, has obvious advantages in reducing dependence upon oil for fuel. In addition, a vehicle burning a ninety percent gasoline and ten percent ethanol mixture produces about a fourth less carbon dioxide.

For a variety of reasons, ethanol is currently being produced in quantity largely from grains such as corn or wheat. Such grains naturally contain high concentrations of starches. In the process of converting grains to ethanol, such starches are ordinarily converted to sugars using a number of readily available enzymes. Ethanol is processed from these sugars, chiefly glucose also known as C6 sugar, using a fermentation process. Although there are other processes, this is currently the most common method of producing ethanol from grains. Under the current state of the art, about 92 gallons of ethanol may be produced from a ton of corn.

Ethanol may also be produced from biomass which is considered to be any naturally occurring organic material containing cellulose. For purposes of this discussion, biomass refers to wood waste including slash, pine needles, sawdust, and any other currently unwanted wood material; but biomass could also include any organic material including cellulose. Some ethanol is currently being produced from biomass, but such production is currently significantly more expensive and less efficient than production of ethanol from grains.

Biomass contains cellulose and hemicellulose which may be converted into C6 sugars such as glucose and C5 sugars such as xylose. The structure of these materials in biomass may be considered as a long strand of crystalline cellulose surrounded by a layer of hemicellulose with both the cellulose and hemicellulose surrounded by a layer of what is known as lignin. Hemicelluloses are generally linear or branched polymers of C5 sugars, but may include other compounds. Lignin is a polymetric matrix of aromatic structures.

Because of differences in the bonding of compounds in biomass and because of the presence of the lignin sheath, it is much more difficult to process the cellulose and hemicellulose in biomass than it is to process the starches in grains. Most often, an acid hydrolysis process is currently used to extract and reduce the hemicellulose and cellulose to C5 and C6 sugars. Because the process uses sulfuric acid, process equipment such as pumps and pipes must be corrosion resistant and are much more expensive than those used to process grains. The sulfuric acid process also generates a neutralization byproduct, calcium sulfate or gypsum, which must be disposed of. Using the acid hydrolysis process, about 58 gallons of ethanol can be produced from a ton of biomass. Prices vary, of course, but a ton of biomass delivered to a processing site costs approximately one half as much as a ton of grain delivered to a processing site. Even though the feed stock costs much less, acid hydrolysis of biomass to ethanol is not generally economically feasible; because the plant costs are higher than producing ethanol from grains and the yields are lower.

Several methods of pretreating biomass to avoid acid processing have been investigated. The patent to Ladisch et al. (U.S. Pat. No. 5,846,787; Dec. 8, 1998) discloses a process in which cellulose containing material is pretreated by combining the material with water in a reactor and heating the resultant combination to from 160 degrees C. to 220 degrees C. while maintaining the pH at from 5 to 8. The resultant material may then be hydrolyzed using enzymes.

The instant invention is believed to solve, in a unique and effective manner, a variety of problems relating to the use of biomass for production of ethanol. The invention is a method involving an extrusion reactor to break down the biomass physically such that it may be processed enzymatically to produce ethanol.

The ideal pretreatment of biomass for ethanol production should pretreat biomass such that the cellulose and hemicellulose contained within the biomass may easily and efficiently be converted to ethanol using enzymatic hydrolysis. The ideal pretreatment of biomass for ethanol production should allow the conversion of biomass to ethanol without the need for corrosion resistant equipment necessary for acid hydrolysis and similar processes. The ideal pretreatment of biomass for ethanol production should also use materials which are inexpensive, easily handled, and environmentally safe.

### SUMMARY OF THE INVENTION

The pretreatment of biomass for ethanol production of the instant invention is a method of pretreating biomass such as wood waste such that the biomass may be converted to ethanol using enzyme hydrolysis rather than acid hydrolysis. Feed stock may be any cellulose containing organic material, but sawdust or wood chips are used in the following example.

Dry wood chips having a moisture content of forty to fifty percent by weight are introduced into the upstream end of an auger driven extruder through a feed bin. The extruder is a conventional twin screw extruder readily available from a variety of manufacturers. The auger is turned forcing the wood chips downstream. The twin screws of the extruder not only force the wood chips through the extruder, but also act upon the wood chips to change the physical structure of the wood chips. The wood chips are expelled from the extruder in the normal fashion and captured in an output bin.

One of the major objects of the present invention is to pretreat biomass such that the cellulose and hemicellulose contained within the biomass may easily and efficiently be converted to ethanol using enzymatic hydrolysis.

Another objective of the present invention is the pretreatment of biomass for ethanol production which allows the conversion of biomass to ethanol without the need for corrosion resistant equipment necessary for acid hydrolysis and similar processes.

Another objective of the present invention is to use materials which are inexpensive, easily handled, and environmentally safe.

These and other features of the invention will become apparent when taken in consideration with the following detailed description and the drawings.

### BRIEF DESCRIPTION OF THE DRAWINGS

FIG. 1 is a schematic of the pretreatment of biomass for ethanol production of the instant invention.

### DESCRIPTION OF A PREFERRED EMBODIMENT

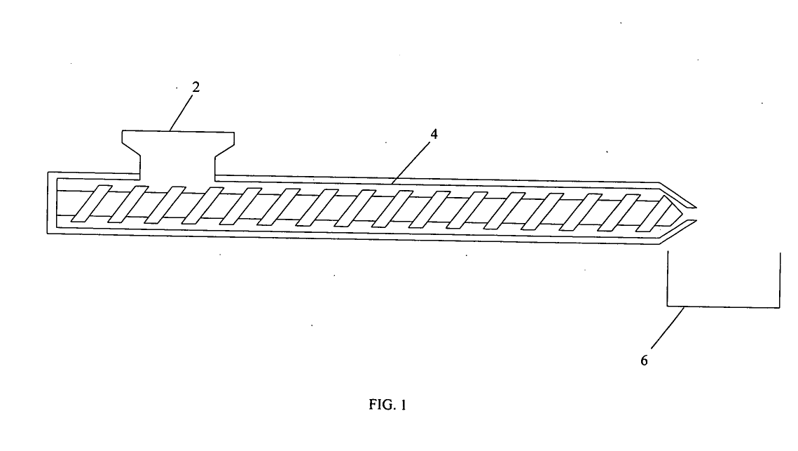

Referring to the drawing, FIGS. 1, a preferred embodiment of the pretreatment of biomass for ethanol production of the instant invention is shown.

Referring now to FIG. 1, wood chips or sawdust (or other lignocellulosic feed stocks) are introduced into a feed bin 2. The wood chips pass through the feed bin 2 and enter a twin screw extruder 4. The screws in the twin screw extruder 4 act upon the wood chips to force them through said twin screw extruder 4 and out the end of said twin screw extruder 4 opposite said feed bin 2. The wood chips leave said twin screw extruder 4 and drop into an output bin 6. The wood chips may then be processed by any of a number of conventional processes to produce ethanol or other desired product. The enzymatic hydrolysis of the wood chips to produce ethanol would be a preferred use of the wood chips pretreated by the extruding process described above.

Hundreds of tests were run to determine the amount of sugars available for hydrolysis under a variety of conditions. All test samples started as sawdust (or wood chips) from Black Hills (South Dakota) Ponderosa Pine. The tests were directed toward determining the percent of the total available glucose which could be recovered from each test sample. That is, glucose was recovered from test samples using conventional enzymatic hydrolysis and the amount of glucose recovered compared with the total amount of glucose known to be present in Ponderosa Pine.

Control samples were untreated sawdust. Extruder samples were sawdust from the same source run through a conventional twin screw extruder. Flour samples were Ponderosa Pine sawdust which had been pulverized to the approximate consistency of flour in a conventional hammer mill. The tests were run to determine whether there was a statistically significant increase in glucose recovery through extrusion. The flour was tested to determine whether any increase in glucose recovery could be attributable to reduction in particle size or whether extrusion had some effect on glucose recovery other than reduction in particle size.

Most of the control sample sawdust particles were in the size range of 0.04 to 0.08 inches. It was somewhat difficult to exactly determine particle size after extrusion because particles tended to clump together, but most of the particles appeared to be between 0.04 and 0.08 inches with 60 percent of the particles being greater than 0.033 inches. The flour had passed through a 0.033 inch screen and the wood particles which made up the flour were, therefore, that size or smaller. Size classification of the flour particles indicated that 50 percent were between 0.004 and 0.008 inches and 23 percent were less than 0.003 inches.

Glucose recovery was measured according to a standard procedure of the National Renewable Energy Laboratory in Colorado referred to as lap-009.

The control sample sawdust average recovery was 3.09 percent with a margin of error of plus or minus 0.38 percent. That is, 3.09 percent of the available glucose was recoverable from untreated sawdust. The flour sample of pulverized sawdust showed an average recovery of 7.69 percent with a margin for error of plus or minus 0.15 percent. Glucose recovery in the extruder samples was done under two sets of conditions with the results of Table A below coming from the extruder operated at a relatively high feed rate and the results of Table B below coming from the same extruder operating at the same rpm with a feed rate of half that of the conditions reflected in Table A.

TABLE A

| Runs | Percent Recovery |
|---|---|
| 1 | 25 |
| 6 | 30 |
| 17 | 35 |
| 8 | 40 |
| 5 | 45 |

TABLE B

| Runs | Percent Recovery |
|---|---|
| 3 | 50 |
| 6 | 55 |
| 5 | 60 |
| 2 | 65 |

For example, under the conditions reflected in Table A, 17 of the 37 total runs showed a glucose recovery of 35 percent and of the 16 total runs under the conditions reflected in Table B, 6 of the runs showed glucose recovery of 55 percent. Although there may be other reasons for the variations in glucose recovery reflected above, it appears likely that the variations are caused differences in the water content of the samples and by variations in the bark and pine needle content in the samples. From the above data it is apparent that milling sawdust particles to a very small size results in a little more than double the glucose recovery. However, pretreating the sawdust by running it through an extruder increases the glucose recovery by more than ten times even though the resulting particles are significantly larger than those in the flour sample.

The control, extruded, and flour particles were scanned with a scanning electron microscope and the following micrographs produced.

[image]

[image]

All three of the pictures, Picture A, Picture B, and Picture C, show wood particles magnified 840 times. Picture A shows typical particles of wood which had been processed using extrusion as described above. Picture B shows wood flour particles of the type referenced above. Picture C shows unprocessed sawdust particles of the type and size which were used in the experiments described above. Picture C indicates that even though the sawdust was produced by sawing and creating particles which were very small compared to the original source of the wood, the regular structure of the wood is still very apparent. That is, sawing may create relatively small pieces of wood, but does not appear to greatly change the basic structure of the wood. As shown in Picture B, impact milling of sawdust make the particles significantly smaller and does appear to cause some disruption of the structure of the wood particles. Examination of Picture A which shows wood particles after extrusion appears to disclose a tremendous disruption of the structure of the wood particles. A comparison of Picture A with Picture B seems to show that there is a much greater disruption of the structure of the wood with extrusion than there was with impact milling. This may explain the much better glucose recovery from samples which had been treated with extrusion than in the samples of flour which had been subjected to impact milling.

While preferred embodiments of this invention have been shown and described above, it will be apparent to those skilled in the art that various modifications may be made in these embodiments without departing from the spirit of the present invention.

We claim:

1. A method for pretreating biomass resulting in a significant increase in sugar recovery from such biomass comprising:
Pretreating the biomass by processing said biomass through a twin screw extruder prior to hydrolysis.
